(19) United States
(12) Patent Application Publication
(10) Pub. No.: US 2021/0238484 A1
(43) Pub. Date: Aug. 5, 2021

(54) BULK-METAL CRYSTALLINE TRANSITION METAL BASED HETEROGENEOUS CATALYSTS, METHODS OF MAKING AND USES THEREOF

(71) Applicant: SABIC Global Technologies B.V., Bergen op Zoom (NL)

(72) Inventors: Muhammad H. HAIDER, Riyadh (SA); Yasser T. AL-HARBI, Riyadh (SA); Ahmed S. AL-ZENAIDI, Riyadh (SA)

(73) Assignee: SABIC Global Technologies B.V., Bergen op Zoom (NL)

(21) Appl. No.: 17/250,074

(22) PCT Filed: Jun. 4, 2019
(86) PCT No.: PCT/IB2019/054642

§ 371 Date: Nov. 19, 2020

Related U.S. Application Data
(60) Provisional application No. 62/680,725, filed on Jun. 5, 2018.

(51) Int. Cl. C10G 2/00; B01J 23/78; B01J 23/889; B01J 37/08; B01J 37/04; B01J 37/03; B01J 35/00; B01J 37/06; B01J 37/12

(52) U.S. Cl. CPC C10G 2/332; B01J 23/78; B01J 23/8892; B01J 37/082; B01J 37/04; B01J 37/035; C10G 2300/4018; B01J 37/06; B01J 37/12; C10G 2300/1022; C10G 2400/20; C10G 2300/4006; C10G 2300/4012; B01J 35/0006

(57) ABSTRACT

Bulk-metal crystalline catalysts for conversion of synthesis gas to olefins are described. Also described are method of making the catalyst. A bulk metal catalyst can include a first transition metal core surrounded by a silica-alkaline earth metal framework crystal lattice and includes at least one transition metal atoms bound to periphery of the framework crystal lattice. The two transition metals can be iron (Fe), cobalt (Co), manganese (Mn), rhodium (Rh), ruthenium (Ru) and combinations thereof.

### CROSS REFERENCE TO RELATED APPLICATIONS

This application claims the benefit of priority to U.S. Provisional Patent Application No. 62/680,725, filed Jun. 5, 2018, the contents of which are hereby incorporated by reference in their entirety.

### BACKGROUND OF THE INVENTION

### A. Field of the Invention

The invention generally concerns bulk-metal crystalline catalysts that can include a first transition metal core surrounded by a silica-alkaline earth metal framework crystal lattice and includes at least one transition metal atoms bound to periphery of the framework crystal lattice. The two transition metals can be iron (Fe), cobalt (Co), manganese (Mn), rhodium (Rh), ruthenium (Ru) and combinations thereof.

### B. Description of Related Art

Conventional iron (Fe) and cobalt (Co) based catalysts can have advantages over other transition metal (e.g., nickel, copper, cerium, rhodium, ruthenium, etc.) based catalysts for conversion of synthesis gas (“syngas”) to olefins via a Fischer Tropsch process. Synthesis gas is a mixture of hydrogen (H₂) and carbon monoxide (C), and optional carbon dioxide (CO₂). In this process, carbon monoxide and hydrogen in synthesis gas react over a metal-based catalyst to form olefins as shown in the following reactions:

CO+M→M-CO(CO adsorption); (1)

M-CO→M-C+M-O(CO dissociation); (2)

M-C+[H]→M-CH+[H]→M-CH₂;(H₂ adsorption/methylene formation) (3)

M-CH₂+[H]→M-CH₃→M - - - CH₂—CH₃ (Chain propagation/termination in H₂) (4)

where M is a metal atom of the catalyst, [H] is hydrogen atom of the hydrogen gas in synthesis gas, and CH is an intermediate for a methylene group. The advantages of using a Fischer-Tropsch reaction for production of olefins has been attributed to the easy dissociative adsorption of CO, reasonable hydrogen adsorption, easy reducibility of metal oxide surface species and economically feasible (availability and cost). Further advantages include cobalt being stable for long durations and iron being very active and having high water-gas shift (WGS) activity, which can be required for low hydrogen based feedstocks. However, both catalyst have disadvantages. For example, cobalt based catalysts can have low WGS activity and can produce long straight chain products whereas iron based catalysts can have higher WGS activity and produce short chain products. However iron based catalysts can have a short lifetime due to its strong ability to form carbon deposits leading to deactivation.

To address these disadvantages of Fischer-Tropsch catalysts, promotion of Co with manganese (Mn) and/or Fe have been described. Promotion with Mg can shift the reaction towards a more olefinic product distribution as compared to unpromoted cobalt, while addition of iron can promote higher WGS activity. By way of example, U.S. Pat. No. 9,545,620 to Karim et al. describes a catalyst that includes Co, Mg, Fe, and hydrophilic silica as a binder for the production of olefins from syngas. While this catalyst had high conversion of syngas, it produced undesirable quantities of methane (e.g., greater than 30 mol. %). In another example, Sainna et al. (Journal of Thermodynamics & Catalysis, 2016, 7, 172) describes a mesoporous silica support impregnated with Co, Fe, and Mg.

While various supported Co, Fe, Mg promoted catalysts are known, these catalyst suffer in high selectivity to methane and/or involve complicated methodology to prepare.

### SUMMARY OF THE INVENTION

A solution to some of the problems discussed above concerning Fischer-Tropsch conversion of syngas to olefins has been discovered. The solution is premised on a bulk metal crystalline catalyst that is a bimetallic catalyst or a multimetallic (e.g., 3 or more catalytic metals) catalyst. The catalyst can include a first catalytic active transition metal core (e.g., Fe) surrounded by a silica-alkaline earth metal framework crystal lattice that can include at least one additional catalytic active transition metals (e.g., Co, manganese (Mn), rhodium (Rh), ruthenium (Ru) or combinations thereof) bound to the periphery of the framework crystal lattice. A non-limiting example of a bimetallic bulk metal crystalline catalyst would be a CoFeSiMgO type catalyst. A non-limiting example of a trimetallic bulk metal crystalline catalyst would be a MnCoFeSiMgO type catalyst. Notably, and as exemplified in non-limiting Examples, the catalyst can be stable for very long time duration (e.g., 400 hours or more) and/or can produce short chain products (e.g., C2-C4 olefinic products) along with high WGS activity. Without wishing to be bound by theory, it is believed that putting the Fe metal in the core of the crystal lattice inhibits deactivation of the catalyst.

In one aspect of the current invention, bulk metal crystalline catalysts are described. A bulk-metal crystalline catalyst can include a first transition metal core surrounded by a silica-alkaline earth metal framework crystal lattice that can include at least one transition metal atom bound to periphery of the framework crystal lattice. The transition metals can include iron (Fe), cobalt (Co), manganese (Mn), rhodium (Rh), ruthenium (Ru) and combinations thereof. The first transition metal can be Fe. The catalyst can have a formula of (M¹)ₐ(Fe)_{c}SiₓM³_{y}O_{z} where M¹ is a transition metal selected from the group consisting of cobalt, ruthenium, rhodium manganese, and combinations thereof M³ is an alkaline earth metal, and 0.01≤a<1 or, 0.01≤c<1, 0.03≤x<1, 0.26≤y<1, and z is balances the valence of the catalyst. The catalyst can include at least two transitions metals and have a formula of (M¹)ₐ(M²)_{b}(Fe)_{c}SiₓM³_{y}O_{z} where M¹ and M² are the at least two transition metals selected from the group consisting of cobalt, ruthenium, rhodium manganese, and combinations thereof M³ is an alkaline earth metal, and 0.01≤a<1, 0.07≤b<1, 0.01≤c<1, 0.03≤x<1, 0.26<y≤1, and z is balances the valence of the catalyst. In some embodiments, z is 0.30 to 0.40, or 0.35 to 0.36. The alkaline earth metal (M³) can include magnesium, calcium, strontium, barium or oxides and mixtures thereof, preferably magnesium. In some embodiments, the first transition metal core is Fe metal surrounded by a silica-magnesia framework crystal lattice and includes cobalt (Co) and manganese (Mn) atoms bound to periphery of the framework crystal lattice. Such a catalyst can have a formula of (Mn)ₐ(Co)_{b}Fe_{c}Siₓ(Mg)_{y}O_{z} where 0.01≤a<1, 0.07≤b<1, 0.01≤c<1, 0.03≤x<1, 0.26<y≤1, and z is 0.3 to 0.4, preferably 0.35. In some instances, the catalyst is absent, or substantially absent (i.e., less than 1 wt. %, or 0 wt. %) a binder.

In another aspect of the invention, methods for preparing the catalyst of the present invention are described. A method can include the steps of: (a) obtaining a solution of a silicon precursor material (e.g., tetra-alkyl silicate such as tetraethyl orthosilicate (TEOS), an alkaline earth metal precursor material (e.g., magnesium chloride) and at least one transition metal precursor materials (e.g., iron citrate, cobalt nitrate, magnesium nitrate, etc.); (b) precipitating a silica/alkaline-earth metal/transition metals agglomerate from the solution, the silica/alkaline-earth metal/transition metals agglomerate can include the first transition metal core bound to a silica-alkaline earth metal framework crystal lattice and the second transition metal on the periphery of the framework crystal lattice and precursor material; and (c) contacting the precipitated material with an oxidizing agent (e.g., hydrogen peroxide (H₂O₂) to remove the precursor material and produce a bulk metal crystalline catalyst having a silica-alkaline earth metal crystal lattice having the first transition metal in the core of the crystal lattice and the second transition metal atom on the periphery of the crystal lattice. A third transition metal can be added to the step (b) dispersion and then an alkaline solution can be added to precipitate a silica/alkaline-earth metal/transition metals agglomerate that includes the first transition metal core bound to a silica-alkaline earth metal framework crystal lattice and the second and third transition metals on the periphery of the framework crystal lattice and precursor material. Prior to step (c), the precipitated material can be isolated and dried at a temperature of 100 C to 150° C., preferably 130° C. The dried material can be calcined at a temperature of 300° C. to 550° C., preferably 450° C. Step (b) precipitation can include adding an alkaline solution comprising ammonia, preferably 7 M ammonia to the solution.

In yet another aspect of the present invention, methods of producing olefins from synthesis gas are described. A method can include contacting a reactant feed that includes hydrogen (H₂) and carbon monoxide (CO) with the catalyst(s) of the present invention, or made by the methods of the present invention, under conditions sufficient to produce an olefin. Conditions can include temperature (e.g., 230° C. to 400° C., preferably, 240° C. to 350° C.), weighted hourly space velocity (WHSV) (e.g., 1000 h⁻¹ to 3000 h⁻¹, preferably 1500 h⁻¹ to 2000 h⁻¹), pressure (e.g., 0.1 MPa to 1 MPa, preferably 0.5 MPa), or combinations thereof. A molar ratio of H₂ to CO can be 1:1 to 10:1, preferably 2:1. The olefin selectivity of the catalyst can be at least 15 mol. %, preferably 20 mol. %, the olefin conversion can be at least 30 mol. %, preferably at least 70 mol. %, more preferably at least 90 mol. %, and the methane selectivity can be less than 30 mol. %, or combinations thereof.

The following includes definitions of various terms and phrases used throughout this specification.

An alkyl group is linear or branched, substituted or substituted, saturated hydrocarbon. Non-limiting examples of alkyl group substituents include alkyl, halogen, hydroxyl, alkyloxy, haloalkyl, haloalkoxy, carboxylic acid, ester, amine, amide, nitrile, acyl, thiol and thioether.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “bulk metal crystalline catalyst” as that term is used in the specification and/or claims, means that the catalyst includes one metal, and does not require a carrier or a support.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the catalysts of the present invention are their abilities to catalyze production of olefins from synthesis gas.

In the context of the present invention at least twenty embodiments are now described. Embodiment 1 is a bulk-metal crystalline catalyst. The bulk catalyst includes a first transition metal core surrounded by a silica-alkaline earth metal framework crystal lattice and including at least one transition metal atom bound to the periphery of the framework crystal lattice, wherein the transition metals are selected from the group consisting of iron (Fe), cobalt (Co), manganese (Mn), rhodium (Rh), ruthenium (Ru) and combinations thereof. Embodiment 2 is the bulk-metal crystalline catalyst of embodiment 1, wherein the first transition metal core contains iron (Fe). Embodiment 3 is the bulk-metal crystalline catalyst of embodiment 2, wherein the catalyst has a formula of (M¹)ₐ(Fe)_{c}SiₓM³_{y}O_{z} where M¹ is the transition metal selected from the group consisting of cobalt, ruthenium, rhodium manganese, and combinations thereof M³ is an alkaline earth metal, and 0.01<a<1 or, 0.01≤c<1, 0.03≤x<1, 0.26≤y<1, and z is balances the valence of the catalyst. Embodiment 4 is the bulk metal crystalline catalyst of embodiment 2, wherein the catalyst has a formula of (M¹)ₐ(M²)_{b}(Fe)_{c}SiₓM³_{y}O_{z} where M¹ and M² are the transitions metals selected from the group consisting of cobalt, ruthenium, rhodium manganese, and combinations thereof M³ is an alkaline earth metal, and 1<a≤0.01, 1<b≤0.07, 1<c≤0.01, 1<x≤0.03, 1<y≤0.26, and z is balances the valence of the catalyst. Embodiment 5 is the bulk-metal crystalline catalyst of any one of embodiments 1 to 4, wherein the alkaline earth metal (M³) is selected from the group consisting of magnesium, calcium, strontium, barium or oxides and mixtures thereof. Embodiment 6 is the bulk-metal crystalline catalyst of embodiment 5, wherein the alkaline earth metal is magnesium. Embodiment 7 is the bulk-metal crystalline catalyst of embodiment 1, wherein first transition metal core is Fe metal surrounded by a silica-magnesia framework crystal lattice and includes cobalt (Co) and manganese (Mn) atoms bound to periphery of the framework crystal lattice. Embodiment 8 is the bulk-metal crystalline catalyst of embodiment 7, wherein the catalyst has a formula of (Mn)ₐ(Co)_{b}Fe_{c}Siₓ(Mg)_{y}O_{z} where 0.01≤a<1, 0.07≤b<1, 0.01≤c<1, 0.03≤x<1, 0.26<y≤1, and z is balances the valence of the catalyst. Embodiment 9 is the bulk-metal crystalline catalyst of any one of embodiments 1 to 8, wherein the catalyst is absent a binder.

Embodiment 10 is a method for preparing the bulk metal crystalline catalyst of any one of embodiments 1 to 9. The method includes the steps of (a) obtaining a solution of a silicon precursor material, an alkaline earth metal precursor material and at least two transition metal precursor materials; (b) precipitating a silica/alkaline-earth metal/transition metals agglomerate from the solution, the silica/alkaline-earth metal/transition metals agglomerate containing the first transition metal core bound to a silica-alkaline earth metal framework crystal lattice and the second transition metal on the periphery of the framework crystal lattice and precursor material; and (c) contacting the precipitated material with an oxidizing agent to remove the precursor material and produce a bulk metal crystalline catalyst having a silica-alkaline earth metal crystal lattice having the first transition metal in the core of the crystal lattice and the second transition metal atom on the periphery of the crystal lattice. Embodiment 11 is the method of embodiment 10, further including the step of adding a third transition metal to the step (b) dispersion and then adding an alkaline solution to precipitate a silica/alkaline-earth metal/transition metals agglomerate containing the first transition metal core bound to a silica-alkaline earth metal framework crystal lattice and the second and third transition metals on the periphery of the framework crystal lattice and precursor material. Embodiment 12 is the method of any one of embodiments 10 and 11, further including the step of isolating and drying the precipitated material at a temperature of 100 C to 150° C., preferably 130° C. prior to step (c). Embodiment 13 is the method of any one of embodiments 10 to 12, further including the step of isolating and drying the crystalline material of step (c) 100 C to 150° C., preferably 130° C. Embodiment 14 is the method of embodiment 13, further including the step of calcining the dried material at 300° C. to 550° C., preferably 450° C. Embodiment 15 is the method of any one of embodiments 10 to 14, wherein step (b) includes adding an alkaline solution containing ammonia, preferably 7 M ammonia to the solution, the oxidizing solution in step (c) hydrogen peroxide (H₂O₂), or both. Embodiment 16 is the method of any one of embodiments 10 to 15, wherein the precursor materials are selected from the group consisting of iron citrate, magnesium chloride, tetra-alkyl silicate, cobalt nitrate, and manganese nitrate.

Embodiment 17 is a method of producing olefins from synthesis gas, the method including the steps of contacting a reactant feed containing hydrogen (H₂) and carbon monoxide (CO) with the catalyst of any one of embodiments 1 or made by the method of any one of embodiments 9 to 16, under conditions sufficient to produce an olefin. Embodiment 18 is the method of embodiment 17, wherein the conditions include a temperature from 230° C. to 400° C., preferably, 240° C. to 350° C., a weighted hourly space velocity of 1000 h⁻¹ to 3000 h⁻¹, preferably 1200 h⁻¹ to 2000 h⁻¹, a pressure of 0 to 1 MPa, preferably 0.5 MPa, or combinations thereof. Embodiment 19 is the method of any one of embodiments 17 and 18, wherein a molar ratio of H₂ to CO is 1:1 to 10:1, preferably 2:1. Embodiment 20 is the method of any one of embodiments 17 and 19, wherein the olefin selectivity is at least 15 mol. %, preferably 20 mol. %, the olefin conversion is at least 30 mol. %, preferably at least 70 mol. %, more preferably at least 90 mol. %, and the methane selectivity is less than 30 mol. %, or combinations thereof.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein. Furthermore, compositions of the invention can be used to achieve methods of the invention.

### BRIEF DESCRIPTION OF THE DRAWINGS

Advantages of the present invention may become apparent to those skilled in the art with the benefit of the following detailed description and upon reference to the accompanying drawings.

FIG. 1 depicts an image of a bimetallic bulk metal crystalline catalyst (CoFeSiMgO) of the present invention.

FIG. 2 depicts an image of a trimetallic bulk metal crystalline catalyst (MnCoFeSiMgO) of the present invention.

While the invention is susceptible to various modifications and alternative forms, specific embodiments thereof are shown by way of example in the drawings. The drawings may not be to scale.

### DETAILED DESCRIPTION OF THE INVENTION

A discovery has been made that provides a solution to problems associated with catalysts used in the Fisher-Tropsch process to produce olefins from syngas. The discovery is premised on using a bulk bimetallic or multimetallic crystalline catalyst(s). The crystalline catalyst can includes a first catalytic transition metal core surrounded by a silica-alkaline earth metal framework crystal lattice and including at least one additional catalytic transition metal bound to the periphery of the framework crystal lattice. Non-limiting examples of the transition metal are Fe, Co, Mn, Rh, Ru, and combinations thereof. Preferably, the first transition metal is Fe and the transition metal(s) bound to the periphery of the framework are Co, Mn, Rh, Ru, or combinations thereof. Further, the catalytic activity and stability for the catalyst of the present invention is comparable or better as compared to the conventional catalysts for the Fischer-Tropsch process. Therefore, the catalyst of the present invention provides a technical solution to at least some of the problems associated with the currently available catalysts for the Fischer-Tropsch process mentioned above, such as low selectivity, low catalytic activity, and/or low stability.

These and other non-limiting aspects of the present invention are discussed in further detail in the following sections.

### A. Catalysts of the Present Invention

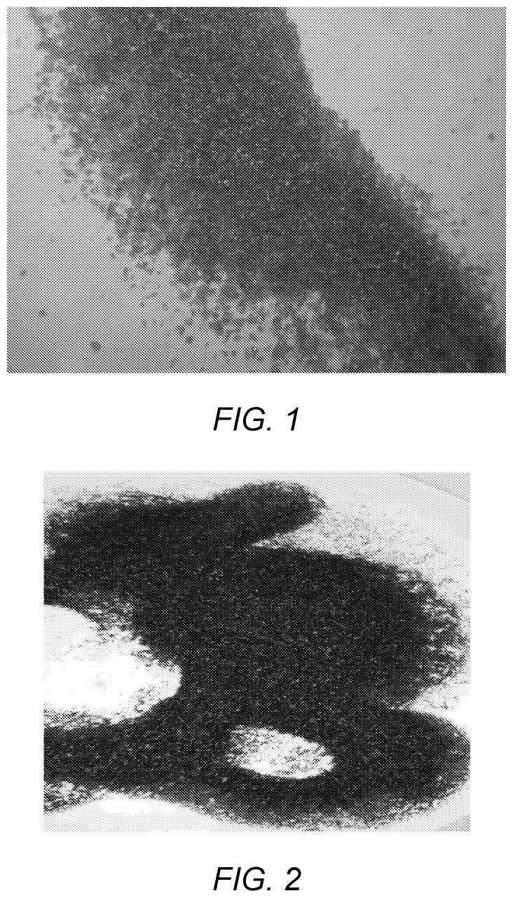

The catalyst of the present invention can be a bulk metal crystalline catalyst. Said another way, the catalyst is not supported or does not include a binder. The catalyst can be a single crystal catalyst (See, for example, FIGS. 1 and 2 of the Examples). The catalyst can include a silica-alkaline earth metal crystal framework that includes at least two transition metals. Non-limiting examples of alkaline earth metals (Column 2 of the Periodic Table) include Mg, Ca, Sr, Ba and combinations thereof. Non-limiting examples of transition metals (Columns 5-12 of the Periodic Table include Mn, Fe, Co, Rh, Ru, chromium (Cr), molybdenum (Mo), tungsten (W), nickel (Ni), palladium (Pd), copper (Cu), silver (Ag), zinc (Zn), cadmium (Cd), and combinations thereof. The catalyst of the present invention can include up to 20 wt. % of the total amount of transition metal, from 0.001 wt. % to 20 wt. %, from 0.01 wt. % to 15 wt. %, or from 1 wt. % to 10 wt. % and all wt. % or at least, equal to, or between any two of 0.001 wt. %, 0.01 wt. %, 0.1 wt. %, 1 wt. %, 2 wt. %, 3 wt. %, 4 wt. %, 5 wt. %, 6 wt. %, 7 wt. %, 8 wt. %, 9 wt. %, 10 wt. %, 15 wt. %, and 20 wt. %, from 0.001 to 1 wt. % alkaline earth metal, with the balance being silicon and oxygen.

The catalyst can have a formula of (M¹)ₐ(Fe)_{c}SiₓM³_{y}O_{z} where M¹ is a transition metal selected from the group consisting of cobalt, ruthenium, rhodium manganese, and combinations thereof M³ is an alkaline earth metal, and 0.01≤a<1 or at least, equal to, or between any two of 0.01, 0.05, 0.1, 0.2, 0.3, 0.4, 0.5, 0.6, 0.7, 0.8, 0.9 and 1; 0.01≤c<1, or at least, equal to, or between any two of 0.01, 0.05, 0.1, 0.2, 0.3, 0.4, 0.5, 0.6, 0.7, 0.8, 0.9 and 1; 0.03≤x<1, or at least, equal to, or between any two of 0.3, 0.4, 0.5, 0.6, 0.7, 0.8, 0.9 and 1; 0.26≤y<1, or at least, equal to, or between any two of 0.26, 0.3, 0.4, 0.5, 0.6, 0.7, 0.8, 0.9 and 1; and z is balances the valence of the catalyst. In some embodiments, z is 0.30 to 0.40, or 0.35 to 0.36, or 0.35 to 0.36.

In some instances, the catalyst can include at least two transitions metals and have a formula of (M¹)ₐ(M²)_{b}(Fe)_{c}SiₓM³_{y}O_{z} where M¹ and M² are the at least two transition metals selected from the group consisting of cobalt, ruthenium, rhodium manganese, and combinations thereof M³ is an alkaline earth metal, and 0.01≤a<1, or at least, equal to, or between any two of 0.01, 0.05, 0.1, 0.2, 0.3, 0.4, 0.5, 0.6, 0.7, 0.8, 0.9 and 1; 0.07≤b<1, or at least, equal to, or between any two of 0.7, 0.8, 0.9 and 1; 0.01≤c<1, or at least, equal to, or between any two of 0.01, 0.05, 0.1, 0.2, 0.3, 0.4, 0.5, 0.6, 0.7, 0.8, 0.9 and 1; 0.03≤x<1, or at least, equal to, or between any two of 0.3, 0.4, 0.5, 0.6, 0.7, 0.8, 0.9 and 1; 0.26<y≤1, or at least, equal to, or between any two of 0.26, 0.3, 0.4, 0.5, 0.6, 0.7, 0.8, 0.9 and 1; and z is balances the valence of the catalyst. In some embodiments, z is 0.30 to 0.40, or 0.35 to 0.36.

In some embodiments, the catalyst is (consists of) a MnCoFeSiMgO bulk metal crystalline catalyst. Such a catalyst can have a formula of (Mn)ₐ(Co)_{b}FeSiₓ(Mg)_{y}O_{z} where 0.01≤a<1, or at least, equal to, or between any two of 0.01, 0.05, 0.1, 0.2, 0.3, 0.4, 0.5, 0.6, 0.7, 0.8, 0.9 and 1; 0.07≤b<1, or at least, equal to, or between any two of 0.7, 0.8, 0.9 and 1; 0.01≤c<1, or at least, equal to, or between any two of 0.01, 0.05, 0.1, 0.2, 0.3, 0.4, 0.5, 0.6, 0.7, 0.8, 0.9 and 1; 0.03≤x<1, or at least, equal to, or between any two of 0.3, 0.4, 0.5, 0.6, 0.7, 0.8, 0.9 and 1; 0.26<y≤1, or at least, equal to, or between any two of 0.26, 0.3, 0.4, 0.5, 0.6, 0.7, 0.8, 0.9 and 1; and z is balances the valence of the catalyst. In some embodiments, z is 0.30 to 0.40, or 0.35 to 0.36.

In some embodiments, the active catalyst can include a molar ratio of cobalt to manganese in a range of 0.05 to 3 and all ranges and values there between including 0.05 to 0.10, 0.10 to 0.20, 0.20 to 0.40, 0.40 to 0.60, 0.60 to 0.80, 0.80 to 1.0, 1.0 to 1.2, 1.2 to 1.4, 1.4 to 1.6, 1.6 to 1.8, 1.8 to 2.0, 2.0 to 2.2, 2.2 to 2.4, 2.4 to 2.6, 2.6 to 2.8, and 2.8 to 3.0. A weight ratio of active metal (cobalt and manganese) to silica (SiO) may be in a range of 0.05 to 5 and all ranges and values there between including 0.05 to 0.10, 0.10 to 0.20, 0.20 to 0.40, 0.40 to 0.60, 0.60 to 0.80, 0.80 to 1.0, 1.0 to 1.2, 1.2 to 1.4, 1.4 to 1.6, 1.6 to 1.8, 1.8 to 2.0, 2.0 to 2.2, 2.2 to 2.4, 2.4 to 2.6, 2.6 to 2.8, 2.8 to 3.0, 3.0 to 3.2, 3.2 to 3.4, 3.4 to 3.6, 3.6 to 3.8, 3.8 to 4.0, 4.0 to 4.2, 4.2 to 4.4, 4.4 to 4.6, 4.6 to 4.8, and 4.8 to 5.0. Overall, the active catalyst may have a composition of 3 to 20 wt. % manganese, 0.05 to 8 wt. % cobalt, 40 to 80 wt. % silica, and 0.05 to 8 wt. % iron. Stability of the active catalyst can be quantified at a conversion rate of 30 to 90 over 100 hours under a temperature of 240 to 350° C.

### B. Preparation of the Bulk Metal Crystalline Catalysts of the Present Invention

The bulk metal crystalline catalysts of the present invention are made using methodology to produce singularly crystalline transition metal based alkaline earth-silicates. The method is such that the alkaline earth metal-silicates are first introduced into an aqueous media in the form of sol, which has certain dimensions in terms of water ligands, alkaline earth metal and silica portion. A first transition metal precursor (e.g., Fe) can act as a chelating agent in the core of the silica-alkaline earth metal sol. Afterwards, the second or more metal precursors can be added under conditions suitable to organize them on the periphery. At this stage, a precipitating agent can be added such that water is removed from this hetero-structure and produce a first transition metal (e.g., Fe) cored metal based alkaline earth metal-silicate which can have precursor material from the first transition material deposited around the framework producing a solid material containing single crystals inside the solid. To release (rejuvenate) the crystals, the precursor material is removed via oxidation of the precursor (e.g., washing with an oxidizing agent) to produce a distinct single crystal bulk metal catalyst. This methodology is in contrast to methods using hydrophilic silica as a binder and/or fumed silica as a support.

According to embodiments of the invention, a method may include providing an alkaline earth metal precursor solution. Non-limiting examples of the alkaline earth metal precursors may include magnesium chloride, magnesium acetate, calcium chloride, strontium chloride, strontium acetate, barium chloride, barium acetate, and combinations thereof. The solution can water. The alkaline earth metal salt solution may have a concentration in a range of 0.1 to 5 M and all ranges and values there between including 0.1 to 0.2 M, 0.2 to 0.4 M, 0.4 to 0.6 M, 0.6 to 0.8 M, 0.8 to 1.0 M, 1.0 to 1.2 M, 1.2 to 1.4 M, 1.4 to 1.6 M, 1.6 to 1.8 M, 1.8 to 2.0 M, 2.0 to 2.2 M, 2.2 to 2.4 M, 2.4 to 2.6 M, 2.6 to 2.8 M, 2.8 to 3.0 M, 3.0 to 3.2 M, 3.2 to 3.4 M, 3.4 to 3.6 M, 3.6 to 3.8 M, 3.8 to 4.0 M, 4.0 to 4.2 M, 4.2 to 4.4 M, 4.4 to 4.6 M, 4.6 to 4.8 M, and 4.8 to 5.0 M. In embodiments of the invention, the alkaline metal salt solution may be continuously stirred under a temperature in a range of 45° C. to 90° C. and all ranges and values there between. The duration for stirring may be in a range of 1 to 5 hours and all ranges and values there between.

A silica precursor material can be added to the alkaline earth metal (M³) solution. In some embodiments, the silica precursor material is added slowly (e.g., dropwise over time). Non-limiting examples of silica precursor material includes tetra-alkyl silicate, diethoxydimethylsilane (DEMS), tetramethyl orthosilicate (TMOS), tetraethyl orthosilicate (TEOS), and combinations thereof. In embodiments of the invention, the tetra-alkyl silicate can be TEOS. According to embodiments of the invention, the first mixture may have a alkaline earth metal to silicon weight (e.g., M³:Si) ratio of 0.3 to 2 and all ranges and values there between including 0.4, 0.5, 0.6, 0.7, 0.8, 0.9, 1.0, 1.1, 1.2, 1.3, 1.4, 1.5, 1.6, 1.7, 1.8, 1.9, and 2.0.

The first transition metal precursor solution can be added to the solution (first mixture) followed by the second transition metal precursor. By way of example, an iron precursor material can be added to the alkaline metal precursor/silica precursor solution, followed by a cobalt precursor material to form a second mixture. Non-limiting examples for iron precursor can include ferric citrate, ferric chloride, and combinations thereof. Non-limiting examples for cobalt precursor can include cobalt nitrate, cobalt acetate, and combinations thereof. In some embodiments, the amount of iron precursor added to the second mixture can be 0.1 to 5 g and all ranges and values there between including 0.1 to 0.5 g, 0.5 to 1.0 g, 1.0 to 1.5 g, 1.5 to 2.0 g, 2.0 to 2.5 g, 2.5 to 3.0 g, 3.0 to 3.5 g, 3.5 to 4.0 g, 4.0 to 4.5 g, 4.5 to 5.0 g. In embodiments of the invention, amount of cobalt precursor material added to the second mixture can be 0.1 to 5 g and all ranges and values there between including 0.1 to 0.5 g, 0.5 to 1.0 g, 1.0 to 1.5 g, 1.5 to 2.0 g, 2.0 to 2.5 g, 2.5 to 3.0 g, 3.0 to 3.5 g, 3.5 to 4.0 g, 4.0 to 4.5 g, 4.5 to 5.0 g. In embodiments of the invention, the second mixture can be stirred for a duration of 0.3 to 5 hrs and all ranges and values there between including 0.3 to 0.5 hrs, 0.5 to 1.0 hrs, 1.0 to 1.5 hrs, 1.5 to 2.0 hrs, 2.0 to 2.5 hrs, 2.5 to 3.0 hrs, 3.0 to 3.5 hrs, 3.5 to 4.0 hrs, 4.0 to 4.5 hrs, 4.5 to 5.0 hrs at a temperature of 20 to 30° C., or about 25° C.

A precipitating agent can be added to the second mixture to form a third mixture. A non-limiting example of the precipitating agent may include ammonia (e.g., 1 to 8 M, or 1, 2, 3, 4, 5, 6, 7, 8 M). In embodiments of the invention, the amount of the precipitating agent added to the third mixture may be in a range of 45 to 100 mL, or and all ranges and values there between, including 45, 50, 55, 60, 65, 70, 75, 80, 85, 90, 95 and 100 mL. The third mixture may be continuously stirred for a duration of 0.5 to 5 hrs including 1, hrs, 2 hrs, 3 hrs, 4 hrs, and 5 hrs at a temperature of 20 to 30° C., or about 25° C. until a gel is obtained. The composition of the third mixture may include 1, 2, 3, 4, 5, 6, 7, or 8 wt. % cobalt, 0.5, 1, 1.5 or 2 wt. % iron, 20, 21, 22, 23, 24, 25, 26, 27, 28, 29 or 30 wt. % magnesium, and 60 to 78.5 wt. % silica and all ranges and values there between.

In embodiments, where an additional transition metal (e.g., a third, fourth, fifth, etc. transition metal) is desired, the additional transition metal precursor(s) can be added to the third mixture to form a fourth mixture. Non-limiting examples of a third transition metal precursor(s) include nitrates, acetates, and combinations thereof of the additional transition metal(s). In some embodiments, the additional transition metal precursor(s) are a magnesium precursor material, a rhodium precursor material, or a ruthenium precursor material. An amount of additional transition metal(s) in the fourth mixture can be 0.1 to 5 g and all ranges and values there between including 0.1 to 0.5 g, 0.5 to 1.0 g, 1.0 to 1.5 g, 1.5 to 2.0 g, 2.0 to 2.5 g, 2.5 to 3.0 g, 3.0 to 3.5 g, 3.5 to 4.0 g, 4.0 to 4.5 g, 4.5 to 5.0 g. The fourth mixture can be agitated at a temperature of 20 to 30° C., or about 25° C. for 0.5 to 5 hrs including 0.5 to 1.0 hrs, 1.0 to 1.5 hrs, 1.5 to 2.0 hrs, 2.0 to 2.5 hrs, 2.5 to 3.0 hrs, 3.0 to 3.5 hrs, 3.5 to 4.0 hrs, 4.0 to 4.5 hrs, 4.5 to 5.0 hrs.

A second quantity of the precipitating agent can be added to the fourth mixture to form a fifth mixture and stirring the fifth mixture until a gel is obtained. According to embodiments of the invention, the second quantity may be 10 to 50 ml of the ammonia solution (1 to 10 M) in the fifth mixture. A fifth mixture can be stirred at a temperature of 20 to 30° C., or about 25° C. for 0.5 to 5 hrs including 0.5 to 1.0 hrs, 1.0 to 1.5 hrs, 1.5 to 2.0 hrs, 2.0 to 2.5 hrs, 2.5 to 3.0 hrs, 3.0 to 3.5 hrs, 3.5 to 4.0 hrs, 4.0 to 4.5 hrs, 4.5 to 5.0 hrs. The composition of the fifth mixture may include 1, 2, 3, 4, 5, 6, 7, or 8 wt. % cobalt, 6, 6.5, 7, 7.5 or 8% manganese, 0.5, 1, 1.5, or wt. % iron, 20, 21, 22, 23, 24, 25, 26, 27, 28, 29, or 30 wt. % magnesium, and 52 to 82.5 wt. % silica.

According to embodiments of the invention, the gel from the third mixture and/or second mixture can be isolated (e.g., filtered or centrifuged), washed with hot water to remove the ammonia, and dried. In embodiments of the invention, the gel may be dried at a temperature of 100 to 150° C. and all values and ranges there between including 100 to 105° C., 105 to 110° C., 110 to 115° C., 115 to 120° C., 120 to 125° C., 125 to 130° C., 130 to 135° C., 135 to 140° C., 140 to 145° C., and 145 to 150° C. The drying process may be 5 to 12 hrs and all ranges and values there between including 6 hrs, 7 hrs, 8 hrs, 9 hrs, 10 hrs, 11 hrs. The dried gel can be contacted with an oxidizing solution (e.g., 50 mL of 10% H₂O₂). In some embodiments, contacting includes immersing the dried gel in an oxidizing solution. Contacting the dried gel with the oxidizing solution removes the remaining precursor material(s) and provides single crystals of the catalytic material.

The crystalline material can be calcined at a temperature of 350 to 600° C. and all ranges and values there between including 350 to 360° C., 360 to 370° C., 370 to 380° C., 380 to 390° C., 390 to 400° C., 400 to 410° C., 410 to 420° C., 420 to 430° C., 430 to 440° C., 440 to 450° C., 450 to 460° C., 460 to 470° C., 470 to 480° C., 480 to 490° C., 490 to 500° C., 500 to 510° C., 510 to 520° C., 520 to 530° C., 530 to 540° C., 540 to 550° C., 550 to 560° C., 560 to 570° C., 570 to 580° C., 580 to 590° C., 590 to 600° C. to produce the bulk metal crystalline catalyst(s) of the present invention. A heating rate for the calcination may be in a range of 1 to 5° C./min and all ranges and values there between including 2° C./min, 3° C./min, and 4° C./min. A calcination duration may be in a range of 2 to 12 hrs and all ranges and values there between including 3 hrs, 4 hrs, 5 hrs, 6 hrs, 7 hrs, 8 hrs, 9 hrs, 10 hrs, and 11 hrs.

C. Method of Producing Olefins from a Reactant Feed that Includes H₂ and CO.

The active catalyst of the present invention can catalyze the conversion of a reactant feed that includes H₂ and CO (e.g., synthesis gas) to produce olefins. Olefins can include olefins have 2, 3, 4, and 5 carbon atoms. For example, C2 to C4 olefins includes hydrocarbons that include 2, 3, 4 carbon atoms. Non-limiting examples of olefins include acetylene, propene, 1-butene, isobutylene, isoprene, and the like.

In embodiments of the invention, the synthesis gas can include 60 to 72 vol. % hydrogen and 28 to 40 vol. % carbon monoxide. In some embodiments, the molar ratio of H₂ to CO can be 1:1 to 10:1, or at least, equal to, or between any two of 1:1, 2:1, 3:1, 4:1, 5:1, 6:1, 7:1, 8:1, 9:1, and 10:1. The reaction conditions can include temperature, pressure and WHSV. The reaction temperature in a range of 240 to 400° C. and all ranges and values there between including 240 to 250° C., 250 to 260° C., 260 to 270° C., 270 to 280° C., 280 to 290° C., 290 to 300° C., 300 to 310° C., 310 to 320° C., 320 to 330° C., 330 to 340° C., 340 to 350° C., 350 to 360° C., 360 to 370° C., 370 to 380° C., 380 to 390° C., and 390 to 400° C. The reaction conditions can include a reaction pressure in a range of 0.1 to 1.0 MPa and all ranges and values there between including 0.2, 0.3, 0.4, 0.5, 0.6, 0.7, 0.8 and 0.9 MPa. In embodiments of the invention, a weight hourly space velocity for the synthesis gas can a range of 1200 to 2500 hr⁻¹, and all ranges and values there between 1200 to 1300 hr⁻¹, 1300 to 1400 hr⁻¹, 1400 to 1500 hr⁻¹, 1500 to 1600 hr⁻¹, 1600 to 1700 hr⁻¹, 1700 to 1800 hr⁻¹, 1800 to 1900 hr⁻¹, 1900 to 2000 hr⁻¹, 2000 to 2100 hr⁻¹, 2100 to 2200 hr⁻¹, 2200 to 2300 hr⁻¹, 2300 to 2400 hr⁻¹, and 2400 to 2500 hr⁻¹. Contact of the reactant gas feed with the catalyst produces a product stream that includes olefins, C2+ paraffins, methane, and carbon dioxide (CO₂) can also be formed. The olefins can include C2 to C4 olefins. The product stream can be separated to produce a C2-C4 olefins stream and a by-product stream. The by-product stream can include paraffins, higher olefins (C5+ olefins), methane, and C02. Separation methods include distillation, membrane separations and the like, which are known in the art.

A conversion rate of the synthesis gas can be at least 30 to 100%, or at least, equal to, or between any two of 30%, 35%, 40%, 50%, 55%, 60%, 65%, 70%, 75%, 80%, 85%, 90%, 95% and 100%. Olefins selectivity can range from 10 to 100, or at least, equal to, or between any two of 10, 15, 20, 25, 30, 35, 40, 45, 50, 55, 60, 65, 70, 75, 80, 85, 90, and 100. C2 to C4 olefins selectivity can range from 10 to 100, or at least, equal to, or between any two of 10, 15, 20, 25, 30, 35, 40, 45, 50, 55, 60, 65, 70, 75, 80, 85, 90, and 100. The methane selectivity can be less than, equal to, or between any two of 30%, 25%, 20%, 15%, 10%, 5%, 1%, and 0%. In some instances, the total olefins selectivity at 240° C. is at least 30%, with the selectivity to C2 to C4 olefins being 20% and the methane selectivity being less than 15% after 413 hours on stream.

The method can also include activating the catalyst prior to contact with the reactant feed. To activate the catalyst a gas stream including a reducing agent (e.g., hydrogen) and a chemically inert gas (e.g., nitrogen) can be contacted with the catalyst (e.g., flow through the catalyst bed) at a temperature of 300 to 350° C. and all ranges and values there between. A molar ratio for reducing gas to inert gas in the gas stream can be about 1:1. A heating rate for the activation may be in a range of 2 to 5° C./min and all ranges and values there between including 3° C./min and 4° C./min. A weight hourly space velocity for the gas stream containing the reducing gas may be in a range of 3200 to 4000 hr-1 and all ranges and values there between including 3200 to 3250 hr⁻¹, 3250 to 3300 hr⁻¹, 3300 to 3350 hr⁻¹, 3350 to 3400 hr⁻¹, 3400 to 3450 hr⁻¹, 3450 to 3500 hr⁻¹, 3500 to 3550 hr¹, 3550 to 3600 hr¹, 3600 to 3650 hr⁻¹, 3650 to 3700 hr⁻¹, 3700 to 3750 hr⁻¹, 3750 to 3800 hr⁻¹, 3800 to 3850 hr⁻¹, 3850 to 3900 hr⁻¹, 3900 to 3950 hr⁻¹, and 3950 to 4000 hr⁻¹.

In embodiments of the invention, an apparatus can be adapted for conversion of synthesis gas to C2 to C4 olefins using the aforementioned active catalyst. The apparatus can include a fixed-bed flow reactor. The apparatus can include a catalyst bed in a fixed-bed flow reactor. The apparatus can also include a housing for containing the catalyst bed. In some embodiments the apparatus can include inlet means for introducing synthesis gas to the catalyst bed. The inlet means can an entrance adapted to receive synthesis gas. Further the apparatus can include an outlet means for removing the product stream comprising C2 to C4 olefins from the apparatus. The outlet means can include an exit adapted to flow the product stream from the housing. In embodiments of the invention, the apparatus can include the catalyst according to embodiments of the invention disposed in the catalyst bed. According to embodiments of the invention, the apparatus may be a fluidized bed reactor, and/or a slurry reactor.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Materials Used

The following lab grade chemicals were used without further purification: manganese (II) nitrate tetrahydrate (>97% purity, SigmaMillipore), cobalt (II) nitrate hexahydrate (>98% purity, SigmaMillipore), magnesium chloride (1 M soln.), tetraethyl orthosilicate (TEOS), ferric citrate, ammonium hydroxide soln., and H₂O₂ soln.

### Example 1

### Synthesis of Bulk Metal Crystalline CoFeSiMgO Catalysts

Magnesium chloride (20 ml of 1 M) was diluted with distilled H₂O to 100 mL and stirred vigorously at 50° C. Afterwards, TEOS (10.4 g) was added to the solution dropwise. Then, ferric citrate crystals (0.25 g) followed by cobalt nitrate (0.25 g) were added, and the mixture was stirred for 2 h. Ammonia (1 M, 100 mL solution) was added slowly to the mixture. The resultant solution was stirred for 2 h. The stirred solution was filtered and washed with hot water two times, and the resulting solid cake was placed in oven to dry overnight at 130° C. After drying, the material was immersed in H₂O₂ (15%, 50 mL) for 1 h, followed by filtration and drying for 4 h at 130° C. The material was then calcined at 450° C./3° C. min⁻¹ for 5 h in air until ready to be tested. Further samples were prepared by using 5 and 7 M ammonia solution. From hereafter, the samples are denoted by symbols “A, B C” for 1, 5 and 7 M ammonia solutions respectively. FIG. 1 depicts an image of the A (CoFeSiMgO) catalyst. The platelet type crystalline morphology is evident in FIG. 1.

### Example 2

### Synthesis of Bulk Metal Crystalline CoMnFeSiMgO Catalysts

Magnesium chloride (20 ml of 1 M) was diluted with distilled H₂O to 100 mL and stirred vigorously at 50° C. Afterwards, TEOS (10.4 g) was added to the solution dropwise. Then, ferric citrate crystals (0.25 g) followed by cobalt nitrate (0.25 g) were added, and the mixture was stirred for 2 h. Ammonia (1 M, 50 mL solution) was added slowly to the mixture. The resultant solution was stirred for 1 h. At this point, Mn salt (1.5 g) was added to the mixture and the solution is stirred for another 1 h followed by addition of ammonia (1 M, 50 mL solution), stirred for 1 h, before filtration and washing with hot water two times. The stirred solution was filtered and washed with hot water two times, and the resulting solid cake was placed in oven to dry overnight at 130° C. After drying, the material was immersed in H₂O₂ (15%, 50 mL) for 1 h, followed by filtration and drying for 4 h at 130° C. The material was then calcined at 450° C./3° C. min⁻¹ for 5 h in air until ready to be tested. Further samples were prepared by using 5 and 7 M ammonia solution. From hereafter, the samples are denoted by symbols “D, E, F” for 1, 5 and 7 M ammonia solutions respectively. FIG. 2 depicts an image of the D (MnCoFeSiMgO) catalyst. The platelet type crystalline morphology is evident in FIG. 2.

### Example 3

### Synthesis of Bulk Metal Crystalline CoMnFeSiMgO Catalysts

Magnesium chloride (20 ml of 1 M) was diluted with distilled H₂O to 100 mL and stirred vigorously at 50° C. Afterwards, TEOS (10.4 g) was added to the solution dropwise. Then, ferric citrate crystals (0.25 g) followed by cobalt nitrate (0.25 g) were added, and the mixture was stirred for 2 h. Ammonia (1 M, 50 mL solution) was added slowly to the mixture. The resultant solution was stirred for 1 h. At this point, Mn salt (1.5 g) was added to the mixture and the solution is stirred for another 1 h followed by addition of ammonia (1 M, 50 mL solution), stirred for 1 h, before filtration and washing with hot water two times. The stirred solution was filtered and washed with hot water two times, and the solid cake was placed in oven to dry overnight at 130° C. After drying, the material was immersed in H₂O₂ (10% 50 ml) for 1 h followed by filtration and drying for 4 h at 130° C. The dried material was then calcined at 450° C./3° C. min⁻¹ for 5 h in air until ready to be tested. Further samples are prepared by using 5 and 7 M ammonia solution. From hereafter, the samples are denoted by symbols “G, H, I” for 1, 5 and 7 M ammonia solutions respectively.

### Example 4

### Synthesis of Bulk Metal Crystalline CoFeSiMgO Catalysts

Magnesium chloride (20 ml of 1 M) was diluted with distilled H₂O to 100 mL and stirred vigorously at 50° C. Afterwards, TEOS (10.4 g) was added to the solution dropwise. Then, ferric citrate crystals (0.25 g) followed by cobalt nitrate (1.5 g) were added, and the mixture was stirred for 2 h. Ammonia (1 M, 100 mL solution) was added slowly to the mixture. The resultant solution was stirred for 2 h. The stirred solution was filtered and washed with hot water two times, and the solid cake was placed in oven to dry overnight at 130° C. After drying, the material was immersed in H₂O₂ (15% 50 ml) for 1 h followed by filtration and drying for 4 h at 130° C. The dried material was then calcined at 450° C./3° C. min⁻¹ for 5 h in air until ready to be tested. Further samples are prepared by using 5 and 7 M ammonia solution. From hereafter, the samples are denoted by symbols “J, K, L” for 1, 5 and 7 M ammonia solutions respectively.

### Example 5

### Synthesis of Bulk Metal Crystalline CoMnFeSiMgO Catalysts

Magnesium chloride (20 ml of 1 M) was diluted with distilled H₂O to 100 mL and stirred vigorously at 50° C. Afterwards, TEOS (10.4 g) was added to the solution dropwise. Then, ferric citrate crystals (0.25 g) followed by cobalt nitrate (1.5 g) were added, and the mixture was stirred for 2 h. Ammonia (1 M, 50 mL solution) was added slowly to the mixture. The resultant solution was stirred for 1 h. At this point, Mn salt (1.5 g) was added to the mixture and the solution is stirred for another 1 h followed by addition of ammonia (1 M, 50 mL solution), stirred for 1 h, before filtration and washing with hot water two times. The stirred solution was filtered and washed with hot water two times, and the solid cake was placed in oven to dry overnight at 130° C. After drying, the material was immersed in H₂O₂ (10% 50 ml) for 1 h followed by filtration and drying for 4 h at 130° C. The dried material was then calcined at 450° C./3° C. min⁻¹ for 5 h in air until ready to be tested. Further samples are prepared by using 5 and 7 M ammonia solution. From hereafter, the samples are denoted by symbols “M, N, O” for 1, 5 and 7 M ammonia solutions respectively.

### Example 6

### Catalyst Activity/Selectivity Evaluation

The catalysts from Example 1-5 were evaluated for the activity and selectivity calculations along with short term as well as long studies of the catalyst stabilities. Prior to activity measurement, all of the catalysts were subjected to activation procedure which was performed at 350° C. with the ramp rate of 3° C. min⁻¹ for 16 h in 50:50 H₂/N2 flow (WHSV: 3600 h⁻¹). Catalytic evaluation was carried out in high throughput fixed bed flow reactor setup housed in temperature controlled system fitted with regulators to maintain pressure during the reaction. The products of the reactions were analyzed through online GC analysis using an Agilent GC (Agilent Scientific Instruments, U.S.A.) with a capillary column equipped with TCD and FID detectors. The evaluation was carried out under the following conditions unless otherwise mentioned elsewhere; 5 bar, WHSV: 1875 h⁻¹, H₂/CO ratio of 2. The mass balance of the reactions is calculated to be 95±5%.

The catalysts all performed the same and representative results for catalyst E (CoMnFeSiMgO) are presented in Table 1. Final catalytic materials were crystalline in nature as shown in FIGS. 1 and 2, the crystal including a silica-magnesia support framework with iron at the core and cobalt and manganese on the periphery. The catalytic materials contain the properties of both cobalt and iron, as catalysts were stable for very long time durations like cobalt and have high WGS activity like iron based catalysts. This was considered to be single crystal based catalysis as the materials have crystalline structures. While representative images of two catalysts are depicted, all the catalysts were crystalline in nature. The catalysts were tested for more than 400 h with no signs of deactivation. A maximum of 90% conversion of syngas was achieved with 20% olefins selectivity with only 5% C5+ in it. The same trend is seen in paraffin selectivity. The testing was done in a range of 240-350° C. temperature. The WGS activity was found to be high at high temperatures with high levels of CO₂ and CH₄. As the temperature lowered, CO₂ and CH₄ was decreased due to low WGS activity.

TABLE 1

| Temperature [° C.] | 350 | 325 | 300 | 280 | 260 | 240 |
|---|---|---|---|---|---|---|
| Hours | 193 | 242 | 266 | 338 | 367 | 417 |
| Conversion (mol. %) | 84 | 86 | 76 | 63 | 41 | 18 |
| | Selectivities (mol. %) | | | | | |
| Olefins | 27 | 27 | 28 | 29 | 34 | 33 |
| Paraffins | 14 | 17 | 19 | 22 | 24 | 28 |
| Methane | 23 | 18 | 15 | 14 | 14 | 20 |
| C₂-C₄ | 12 | 13 | 13 | 14 | 13 | 16 |
| C₂ = C₄ | 23 | 21 | 20 | 20 | 22 | 23 |
| C₅₊₋ | 3 | 5 | 7 | 10 | 15 | 12 |
| C₅₊₌ | 4 | 6 | 8 | 9 | 12 | 10 |
| CO₂ | 36 | 37 | 36 | 32 | 23 | 17 |
| Olefins (% yield) | 22 | 22.3 | 20.9 | 18 | 13.9 | 5.8 |

*Catalysts were tested after activation under the following conditions: WHSV: 1875 h⁻¹, H₂/CO: 2.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A bulk-metal crystalline catalyst comprising a first transition metal core surrounded by a silica-alkaline earth metal framework crystal lattice and including at least one transition metal atom bound to the periphery of the framework crystal lattice, wherein the transition metals are selected from the group consisting of iron (Fe), cobalt (Co), manganese (Mn), rhodium (Rh), ruthenium (Ru) and combinations thereof.

2. The bulk-metal crystalline catalyst of claim 1, wherein the first transition metal core comprises iron (Fe).

3. The bulk-metal crystalline catalyst of claim 2, wherein the catalyst has a formula of (M¹)ₐ(Fe)_{c}SiₓM³_{y}O_{z} where M¹ is the transition metal selected from the group consisting of cobalt, ruthenium, rhodium manganese, and combinations thereof M³ is an alkaline earth metal, and 0.01≤a<1 or, 0.01≤c<1, 0.03≤x<1, 0.26≤y<1, and z is balances the valence of the catalyst.

4. The bulk metal crystalline catalyst of claim 2, wherein the catalyst has a formula of (M¹)ₐ(M²)_{b}(Fe)_{c}SiₓM³_{y}O_{z} where M¹ and M² are the transitions metals selected from the group consisting of cobalt, ruthenium, rhodium manganese, and combinations thereof M³ is an alkaline earth metal, and 1<a≤0.01, 1<b≤0.07, 1<c≤0.01, 1<x≤0.03, 1<y≤0.26, and z is balances the valence of the catalyst.

5. The bulk-metal crystalline catalyst of claim 1 4, wherein the alkaline earth metal (M³) is selected from the group consisting of magnesium, calcium, strontium, barium or oxides and mixtures thereof.

6. The bulk-metal crystalline catalyst of claim 5, wherein the alkaline earth metal is magnesium.

7. The bulk-metal crystalline catalyst of claim 1, wherein first transition metal core is Fe metal surrounded by a silica-magnesia framework crystal lattice and includes cobalt (Co) and manganese (Mn) atoms bound to periphery of the framework crystal lattice.

8. The bulk-metal crystalline catalyst of claim 7, wherein the catalyst has a formula of (Mn)ₐ(Co)_{b}Fe_{c}Siₓ(Mg)_{y}O_{z} where 0.01≤a<1, 0.07≤b<1, 0.01≤c<1, 0.03≤x<1, 0.26<y≤1, and z is balances the valence of the catalyst.

9. The bulk-metal crystalline catalyst of claim 1, wherein the catalyst is absent a binder.

10. A method for preparing the bulk metal crystalline catalyst of claim 1, comprising the steps of:
(a) obtaining a solution of a silicon precursor material, an alkaline earth metal precursor material and at least two transition metal precursor materials;
(b) precipitating a silica/alkaline-earth metal/transition metals agglomerate from the solution, the silica/alkaline-earth metal/transition metals agglomerate comprising the first transition metal core bound to a silica-alkaline earth metal framework crystal lattice and the second transition metal on the periphery of the framework crystal lattice and precursor material; and
(c) contacting the precipitated material with an oxidizing agent to remove the precursor material and produce a bulk metal crystalline catalyst having a silica-alkaline earth metal crystal lattice having the first transition metal in the core of the crystal lattice and the second transition metal atom on the periphery of the crystal lattice.

11. The method of claim 10, further comprising adding a third transition metal to the step (b) dispersion and then adding an alkaline solution to precipitate a silica/alkaline-earth metal/transition metals agglomerate comprising the first transition metal core bound to a silica-alkaline earth metal framework crystal lattice and the second and third transition metals on the periphery of the framework crystal lattice and precursor material.

12. The method of claim 10, further comprising isolating and drying the precipitated material at a temperature of 100 C to 150° C. prior to step (c).

13. The method of any one of claim 10, further comprising isolating and drying the crystalline material of step (c) 100 C to 150° C.

14. The method of claim 13, further comprising calcining the dried material at 300° C. to 550° C.

15. The method claim 10, wherein step (b) comprising adding an alkaline solution comprising ammonia, preferably 7 M ammonia to the solution, the oxidizing solution in step (c) hydrogen peroxide (H₂O₂), or both.

16. The method of claim 10, wherein the precursor materials are selected from the group consisting of iron citrate, magnesium chloride, tetra-alkyl silicate, cobalt nitrate, and manganese nitrate.

17. A method of producing olefins from synthesis gas, the method comprising contacting a reactant feed comprising hydrogen (H₂) and carbon monoxide (CO) with the catalyst of claim 1, under conditions sufficient to produce an olefin.

18. The method of claim 17, wherein the conditions comprise a temperature from 230° C. to 400° C., a weighted hourly space velocity of 1000 h⁻¹ to 3000 h⁻¹, a pressure of 0 to 1 MPa or combinations thereof.

19. The method of claim 17, wherein a molar ratio of H₂ to CO is 1:1 to 10:1, preferably 2:1.

20. The method of claim 17, wherein the olefin selectivity is at least 15 mol. %, preferably 20 mol. %, the olefin conversion is at least 30 mol. %, or combinations thereof.
